SEQUENCE LISTING (10 sequences)

SEQ ID NO: 1 - DNA, 25 bp, Artificial sequence
  synthetic construct; forward primer
ccggagctttcaaagaaggaattct

SEQ ID NO: 2 - DNA, 23 bp, Artificial sequence
  synthetic construct; reverse primer
cccctctctccatcctccataaa

SEQ ID NO: 3 - DNA, 25 bp, Artificial sequence
  synthetic construct; forward primer
accaagagagaaagaagtaggcatg

SEQ ID NO: 4 - DNA, 23 bp, Artificial sequence
  synthetic construct; reverse primer
agcagtctggcggcctcacctgg

SEQ ID NO: 5 - DNA, 22 bp, Artificial sequence
  synthetic construct; forward primer
cccagggacctctctctaatca

SEQ ID NO: 6 - DNA, 20 bp, Artificial sequence
  synthetic construct; reverse primer
gctacaggcttgtcactcgg

SEQ ID NO: 7 - DNA, 20 bp, Artificial sequence
  synthetic construct; forward primer
gtgaaggtcggagtcaacgg

SEQ ID NO: 8 - DNA, 22 bp, Artificial sequence
  synthetic construct; reverse primer
tgatgacaagcttcccgttctc

SEQ ID NO: 9 - RNA, 22 nt, Artificial sequence
  synthetic construct
uagcuuaucagacugauguuga

SEQ ID NO: 10 - RNA, 22 nt, Artificial sequence
  synthetic construct
ucguuaaucggcuauaauacgc

(19) United States
(12) Patent Application Publication
(10) Pub. No.: US 2022/0218682 A1
(43) Pub. Date: Jul. 14, 2022

(54) COMPOSITIONS AND METHODS FOR TREATING CANCER

(71) Applicant: THE UNITED STATES GOVERNMENT AS REPRESENTED BY THE DEPARTMENT OF VETERANS AFFAIRS, Washington, DC (US)

(72) Inventor: Amyn Habib, Dallas, TX (US)

(21) Appl. No.: 17/609,757

(22) PCT Filed: May 8, 2020
(86) PCT No.: PCT/US20/32203

§ 371 Date: Nov. 8, 2021

Related U.S. Application Data
(60) Provisional application No. 62/845,460, filed on May 9, 2019.

(51) Int. Cl. A61K 31/4409; A61K 31/517; A61K 31/5377; A61K 39/395; A61P 35/00

(52) U.S. Cl. CPC A61K 31/4409; A61K 31/517; A61P 35/00; A61K 39/3955; A61K 31/5377

(57) ABSTRACT

Provided herein are pharmaceutical compositions comprising an effective amount of an agent that inhibits EGFR signaling and izoniazid, which are useful for treating cancer. This abstract is intended as a scanning tool for purposes of searching in the particular art and is not intended to be limiting of the present invention.

### CROSS-REFERENCE TO RELATED APPLICATIONS

This Application claims the benefit of U.S. Application No. 62/845,460, filed on May 9, 2019, the contents of which are hereby incorporated by reference in their entirety.

### REFERENCE TO SEQUENCE LISTING

The Sequence Listing submitted May 8, 2020 as a text file named “37759_0251P1_ST25.txt,” created on Apr. 22, 2020, and having a size of 3,083 bytes is hereby incorporated by reference pursuant to 37 C.F.R. § 1.52(e)(5).

### BACKGROUND

Oncogene addiction has been described primarily in cancers that express oncogenes rendered constitutively active by mutation. Constitutive activation results in a continuous and unattenuated signaling that may result in a widespread activation of intracellular pathways and reliance of the cell on such pathways for survival. A subset of NSCLCs harbor EGFR activating mutations that render the receptor constitutively active and oncogene addicted. Lung cancers with activating EGFR mutations exhibit a dramatic initial clinical response to treatment with EGFR tyrosine kinase inhibitors (TKIs), but this is followed by the inevitable development of secondary resistance spurring intensive investigation into resistance mechanisms. Major TKI resistance mechanisms identified in EGFR mutant lung cancer include the emergence of other EGFR mutations such as the T790M mutation that prevent TKI enzyme interaction and activation of other receptor tyrosine kinases such as Met or Axl providing a signaling bypass to EGFR TKI mediated inhibition. Rapid feedback loops with activation of STAT3 have also been invoked to mediated EGFR TKI resistance in lung cancer cells with EGFR activating. However, the STAT3 resistance loop was not found in lung cancer cells with EGFR wild type (EGFRwt) and primary resistance to EGFR TKIs. Multiple additional mechanisms and distinct evolutionary pathways have been invoked to explain secondary resistance to EGFR inhibition in lung cancer. In addition, a subset of patients with EGFR activating mutations do not respond to EGFR inhibition, exhibiting a primary or intrinsic resistance, and various mechanisms have been proposed to account for such resistance.

The most common type of EGFR expressed in lung cancer is EGFRwt (EGFR wild type). EGFRwt expressing tumor cells are not oncogene addicted and are usually resistant to EGFR inhibition. The differential responsiveness of cells with EGFR activating mutations may result from altered downstream signal transduction. EGFR activating mutations result in constitutive signaling and have been shown to be transforming. Compared to EGFRwt, EGFR activating mutations lead to activation of extensive networks of signal transduction that, in turn, lead to dependence of tumor cells on continuous EGFR signaling for survival. This is likely the reason that EGFR inhibition is effective in NSCLC patients with EGFR activating mutations despite the well-documented generation of early adaptive survival responses such as STAT3 in EGFR mutant cells. Increased affinity of mutant EGFR for tyrosine kinase inhibitors has also been reported.

TNF (tumor necrosis factor) is a key mediator of the inflammatory response. Depending on the cellular context, it may play a role in cell death or in cell survival and inflammation-induced cancer. TNF is produced by a variety of tissues and is inducibly expressed in response to inflammatory stimuli such as LPS. TNF binds to its cognate receptors TNFR1 or TNFR2 and activates a number of inflammatory signaling networks. Interestingly, malignant cells are known to produce TNF, as are cells in the microenvironment of tumors and there is experimental evidence from a variety of models that TNF can promote the growth of tumors.

MicroRNAs (miRNAs) are small noncoding RNAs that target coding RNAs and regulate the translation and degradation of mRNAs and may play an important role in cancer. Expression levels of miRNAs are altered in various types of cancer, including lung cancer. EGFR activity can regulate miRNA levels in lung cancer. The expression of five microRNAs (hsa-mir-155, hsa-mir-17-3p, hsa-let-7a-2, hsa-mir-145, and hsa-mir-21) were altered in lung cancer from smokers compared to uninvolved lung tissue and there is evidence from examination of archival tissue and cell culture studies that EGFR activity upregulates the expression of mir-21 while inhibition of EGFR activity downregulates miR-21. Both EGFRwt and mutant activity may regulate miR-21 in lung cancer, although EGFR activating mutants appear to have a stronger effect.

Accordingly, improved methods and compositions for treating cancer are needed.

### SUMMARY

In accordance with the purpose(s) of the invention, as embodied and broadly described herein, the invention, in one aspect, relates to compositions that contain an agent that inhibits EGFR signaling and isoniazid, and methods of making and using same for the treatment of cancer, such as, for example, brain cancer (e.g., including Gliomas such as: Astrocytoma, Brain stem glioma, Ependymoma, Glioblastoma multiforme, Mixed glioma, Oligodendroglioma, Optic nerve glioma, and the like), lung cancer, cervical cancer, ovarian cancer, cancer of CNS, skin cancer, prostate cancer, sarcoma, breast cancer, leukemia, colorectal cancer, colon cancer, head cancer, neck cancer, endometrial and kidney cancer.

Thus, provided herein are methods for treating cancer, in a patient in need thereof, said method comprising administering to said patient an effective amount of an EGFR inhibitor and Isoniazid (INH). The EGFR inhibitor can be selected from the group consisting of: erlotinib, afatinib, Cetuximab, panitumumab, Erlotinib HCl, Gefitinib, Lapatinib, Neratinib, Lifirafenib, HER2-nhibitor-1, Nazartinib, Naquotinib, Canertinib, Lapatinib, AG-490, CP-724714, Dacomitinib, WZ4002, Sapitinib, CUDC-101, AG-1478, PD153035 HCL, pelitinib, AC480, AEE788, AP26113-analog, OSI-420, WZ3146, WZ8040, AST-1306, Rociletinib, Genisten, Varlitinib, Icotinib, TAK-285, WHI-P154, Daphnetin, PD168393, Tyrphostin9, CNX-2006, AG-18, AZ5104, Osimertinib, CL-387785, Olmutinib, AZD3759, Poziotinib, vandetanib, necitumumab,

In particular embodiments, the EGFR inhibitor and TNF inhibitor can combinations selected from the group consisting of: erlotinib and Isoniazid; afatinib and Isoniazid; Cetuximab and Isoniazid; panitumumab and Isoniazid; and Gefitinib and Isoniazid.

In the particular cancers treated herein, the EGFR is either EGFR wild type or contains at least one EGFR activating mutation. In some embodiments, the particular cancer being treated can be selected from the group consisting of: brain cancer (e.g., including Gliomas such as: Astrocytoma, Brain stem glioma, Ependymoma, Glioblastoma multiforme, Mixed glioma, Oligodendroglioma, Optic nerve glioma, and the like), lung cancer, cervical cancer, ovarian cancer, cancer of CNS, skin cancer, prostate cancer, sarcoma, breast cancer, leukemia, colorectal cancer, colon cancer, head cancer, neck cancer, endometrial and kidney cancer. In a particular embodiment, the lung cancer is non-small cell lung cancer. In other embodiments, the cancer is a human epithelial carcinoma, which can be selected from the group consisting of: basal cell carcinoma, squamous cell carcinoma, renal cell carcinoma (RCC), ductal carcinoma in situ (DCIS), and invasive ductal carcinoma.

In a particular embodiment, the particular cancer being treated is resistant to EGFR inhibition; or has previously been determined to have been resistant to EGFR inhibition. The cancer resistant to EGFR inhibition can be non-small cell lung cancer.

Also provided is a method of treating a tumor resistant to EGFR inhibition, in a patient in need thereof, comprising administering Isoniazid in combination with an agent that inhibits EGFR activity.

Also provided is a method for treating cancer in a patient in need thereof, the method comprising administering to the patient an effective amount of an agent that inhibits EGFR signaling, or a pharmaceutically acceptable salt thereof, and isoniazid (INH), or a pharmaceutically acceptable salt thereof.

Also provided herein are pharmaceutical compositions comprising a therapeutically effective amount of an EGFR inhibitor and Isoniazid. The EGFR inhibitor can be selected from the group consisting of: erlotinib, afatinib, Cetuximab, panitumumab, Erlotinib HCl, Gefitinib, Lapatinib, Neratinib, Lifirafenib, HER2-nhibitor-1, Nazartinib, Naquotinib, Canertinib, Lapatinib, AG-490, CP-724714, Dacomitinib, WZ4002, Sapitinib, CUDC-101, AG-1478, PD153035 HCL, pelitinib, AC480, AEE788, AP26113-analog, OSI-420, WZ3146, WZ8040, AST-1306, Rociletinib, Genisten, Varlitinib, Icotinib, TAK-285, WHI-P154, Daphnetin, PD168393, Tyrphostin9, CNX-2006, AG-18, AZ5104, Osimertinib, CL-387785, Olmutinib, AZD3759, Poziotinib, vandetanib, necitumumab, In particular embodiments, the EGFR inhibitor and TNF inhibitor are combinations selected from the group consisting of: erlotinib and Isoniazid; afatinib and Isoniazid; Cetuximab and Isoniazid; panitumumab and Isoniazid; and Gefitinib and Isoniazid.

Also provided herein are pharmaceutical compositions comprising: (a) an agent that inhibits EGFR signaling, or a pharmaceutically acceptable salt thereof (b) isoniazid, or a pharmaceutically acceptable salt thereof and (c) a pharmaceutically acceptable carrier, wherein at least one of the agent that inhibits EGFR signaling and isoniazid is present in an effective amount.

Also provided herein are methods for making a pharmaceutical composition, the method comprising combining: (a) an agent that inhibits EGFR signaling, or a pharmaceutically acceptable salt thereof; (b) isoniazid, or a pharmaceutically acceptable salt thereof; and (c) a pharmaceutically acceptable carrier, wherein at least one of the agent that inhibits EGFR signaling and isoniazid is present in an effective amount.

Although aberrant EGFR signaling is widespread in human cancer, EGFR inhibition is primarily effective only in a subset of NSCLC (non-small cell lung cancer) that harbor EGFR activating mutations. A majority of NSCLCs express EGFR wild type (EGFRwt) and do not respond to EGFR inhibition. Tumor necrosis factor (TNF) is a major mediator of inflammation induced cancer. In accordance with the present invention, it has been demonstrated that a rapid increase in TNF level is a universal adaptive response to inhibition of EGFR signaling in lung cancer cells regardless of whether EGFR is mutant or wild type. EGFR inhibition upregulates TNF by a dual mechanism. First, EGFR signaling actively suppresses TNF mRNA levels by inducing expression of microRNA-21 resulting in decreased TNF mRNA stability. Conversely, inhibition of EGFR activity results in loss of miR-21 and increase in TNF mRNA stability. As a second mechanism, activation of TNF-induced NF-κB activation leads to increased TNF transcription in a feedforward loop. Increased TNF mediates intrinsic resistance to EGFR inhibition, while exogenous TNF can protect oncogene addicted lung cancer cells from a loss of EGFR signaling. Biological or chemical inhibition of TNF signaling renders EGFRwt expressing NSCLC cell lines and an EGFRwt PDX model highly sensitive to EGFR inhibition. In oncogene addicted cells, blocking TNF enhances the effectiveness of EGFR inhibition. In accordance with the present invention, there are provided methods for the combined inhibition of EGFR and TNF as a treatment approach useful for treating human cancers, such as lung cancer (e.g., NSCLC, and the like) patients.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows representative data illustrating TNF protein in medium of brain cancer cells (GBM9) after treatment.

FIG. 2 shows representative data illustrating the percent survival of mice injected with tumor cells intracranially after treatment.

FIG. 3 shows a representative schematic of TNF signaling triggered by EGFR inhibition depicting the adaptive response triggered by EGFR inhibition. Specifically, the left panel indicates that inhibition of EGFR leads to increased TNF mRNA via increased stability of TNF mRNA and increased NF-κB mediated transcription of TNF. Increased TNF leads to NF-κB activation in a feed-forward loop. Activation of NF-κB leads to resistance to EGFR inhibition induced cell death. The right panel shows that blocking the TNF-NF-κB adaptive response renders lung cancer cells sensitive to EGFR inhibition. Etanercept (Enbrel) inhibits TNF signaling at the receptor level while thalidomide inhibits both NF-κB activation and upregulation of TNF. Referring to the bottom panel, upon EGFR inhibition, NF-κB activation and accumulation of TNF form a feedforward loop to enhance each other.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Provided herein are methods for treating cancer, in a patient in need thereof, said method comprising administering to said patient an effective amount of an EGFR inhibitor and Isoniazid.

The EGFR inhibitor can be selected from the group consisting of: erlotinib, afatinib, Cetuximab, panitumumab, Erlotinib HCl, Gefitinib, Lapatinib, Neratinib, Lifirafenib, HER2-nhibitor-1, Nazartinib, Naquotinib, Canertinib, Lapatinib, AG-490, CP-724714, Dacomitinib, WZ4002, Sapitinib, CUDC-101, AG-1478, PD153035 HCL, pelitinib, AC480, AEE788, AP26113-analog, OSI-420, WZ3146, WZ8040, AST-1306, Rociletinib, Genisten, Varlitinib, Icotinib, TAK-285, WHI-P154, Daphnetin, PD168393, Tyrphostin9, CNX-2006, AG-18, AZ5104, Osimertinib, CL-387785, Olmutinib, AZD3759, Poziotinib, vandetanib, and necitumumab.

In particular embodiments, the EGFR inhibitor and TNF inhibitor can combinations selected from the group consisting of: erlotinib and Isoniazid; afatinib and Isoniazid; Cetuximab and Isoniazid; panitumumab and Isoniazid; and Gefitinib and Isoniazid.

In the particular cancers treated herein, the EGFR is either EGFR wild type or contains at least one EGFR activating mutation. In some embodiments, the particular cancer being treated can be selected from the group consisting of: brain cancer (e.g., including Gliomas such as: Astrocytoma, Brain stem glioma, Ependymoma, Glioblastoma multiforme, Mixed glioma, Oligodendroglioma, Optic nerve glioma, and the like), lung cancer, cervical cancer, ovarian cancer, cancer of CNS, skin cancer, prostate cancer, sarcoma, breast cancer, leukemia, colorectal cancer, colon cancer, head cancer, neck cancer, endometrial and kidney cancer. In a particular embodiment, the lung cancer is non-small cell lung cancer. In other embodiments, the cancer is a human epithelial carcinoma, which can be selected from the group consisting of: basal cell carcinoma, squamous cell carcinoma, renal cell carcinoma (RCC), ductal carcinoma in situ (DCIS), and invasive ductal carcinoma.

In a particular embodiment, the particular cancer being treated is resistant to EGFR inhibition; or has previously been determined to have been resistant to EGFR inhibition. The cancer resistant to EGFR inhibition can be non-small cell lung cancer.

Also provided is a method of treating a tumor resistant to EGFR inhibition, in a patient in need thereof, comprising administering Isoniazid in combination with an agent that inhibits EGFR activity.

Also provided herein are pharmaceutical compositions, said compositions comprising a therapeutically effective amount of an EGFR inhibitor and Isonaizid. The EGFR inhibitor can be selected from the group consisting of: erlotinib, afatinib, Cetuximab, panitumumab, Erlotinib HCl, Gefitinib, Lapatinib, Neratinib, Lifirafenib, HER2-nhibitor-1, Nazartinib, Naquotinib, Canertinib, Lapatinib, AG-490, CP-724714, Dacomitinib, WZ4002, Sapitinib, CUDC-101, AG-1478, PD153035 HCL, pelitinib, AC480, AEE788, AP26113-analog, OSI-420, WZ3146, WZ8040, AST-1306, Rociletinib, Genisten, Varlitinib, Icotinib, TAK-285, WHI-P154, Daphnetin, PD168393, Tyrphostin9, CNX-2006, AG-18, AZ5104, Osimertinib, CL-387785, Olmutinib, AZD3759, Poziotinib, vandetanib, and necitumumab. In particular embodiments, the EGFR inhibitor and TNF inhibitor are combinations selected from the group consisting of: erlotinib and Isoniazid; afatinib and Isoniazid; Cetuximab and Isoniazid; panitumumab and Isoniazid; and Gefitinib and Isoniazid.

### A. Definitions

As used in the specification and the appended claims, the singular forms “a,” “an,” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X, and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the phrase “EGFR inhibitor” (also referred to as EGFR TKI) or an “agent that inhibits EGFR activity” refers to any agent (molecule) that functions to reduce or inactivate the biological activity of epidermal growth factor receptor (EGFR). Exemplary EGFR inhibitors include erlotinib, afatinib, Cetuximab, panitumumab, Erlotinib HCl, Gefitinib, Lapatinib, Neratinib, Lifirafenib, HER2-nhibitor-1, Nazartinib, Naquotinib, Canertinib, Lapatinib, AG-490, CP-724714, Dacomitinib, WZ4002, Sapitinib, CUDC-101, AG-1478, PD153035 HCL, pelitinib, AC480, AEE788, AP26113-analog, OSI-420, WZ3146, WZ8040, AST-1306, Rociletinib, Genisten, Varlitinib, Icotinib, TAK-285, WHI-P154, Daphnetin, PD168393, Tyrphostin9, CNX-2006, AG-18, AZ5104, Osimertinib, CL-387785, Olmutinib, AZD3759, Poziotinib, vandetanib, necitumumab, and the like.

As used herein, the phrase “Isoniazid” or “INH” refers to the well-known antibiotic that is commonly used to treat active tuberculosis. Isoniazid corresponds to CAS Registry Number: 54-85-3; and DrugBank Accession Number DB00951.

Exemplary cancers contemplated for treatment herein can be selected from the group consisting of lung cancer, cervical cancer, ovarian cancer, cancer of CNS, skin cancer, prostate cancer, sarcoma, breast cancer, leukemia, colorectal cancer, colon cancer, head cancer, neck cancer, endometrial and kidney cancer. In another aspect, the cancer is selected from the group consisting of non-small cell lung cancer (NSCLC), small cell lung cancer, breast cancer, acute leukemia, chronic leukemia, colorectal cancer, colon cancer, brain cancer, carcinoma, ovarian cancer, or endometrial cancer, carcinoid tumors, metastatic colorectal cancer, islet cell carcinoma, metastatic renal cell carcinoma, adenocarcinomas, glioblastoma multiforme, bronchoalveolar lung cancers, non-Hodgkin's lymphoma, neuroendocrine tumors, and neuroblastoma. In another aspect, the cancer is ovarian, colon, colorectal or endometrial cancer.

The terms “treatment” or “treating” of a subject includes the application or administration of a compound of the invention to a subject (or application or administration of a compound or pharmaceutical composition of the invention to a cell or tissue from a subject) with the purpose of stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indicia of success in the treatment or amelioration of an injury, pathology or condition, including any objective or subjective parameter such as abatement; remission; lessening of the rate of worsening; stabilization, diminishing of symptoms or making the injury, pathology or condition more tolerable to the subject; slowing in the rate of degeneration or decline; making the final point of degeneration less debilitating; or improving a subject's physical or mental well-being. In an embodiment, the term “treating” can include increasing a subject's life expectancy.

The term “in combination with” refers to the concurrent administration of a combination of EGFR and TNF inhibitor compounds; or the administration of either one of the compounds prior to the administration of the other inhibitory compound.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, an “effective amount” of a compound or composition for treating a particular disease, such as cancer, is an amount that is sufficient to ameliorate, or in some manner reduce the symptoms associated with the disease. Such amount can be administered as a single dosage or can be administered according to a regimen, whereby it is effective. The amount can cure the disease but, in certain embodiments, is administered in order to ameliorate the symptoms of the disease. In particular embodiments, repeated administration is required to achieve a desired amelioration of symptoms. A “therapeutically effective amount” or “therapeutically effective dose” can refer to an agent, compound, material, or composition containing a compound that is at least sufficient to produce a therapeutic effect. An effective amount is the quantity of a therapeutic agent necessary for preventing, curing, ameliorating, arresting or partially arresting a symptom of a disease or disorder.

As used herein, the term “individually effective amount” refers to an amount of a single component, e.g., an agent that modulates GalR1, in isolation that is sufficient to achieve the desired result or to have an effect on an undesired condition. For example, an “individually therapeutically effective amount” refers to an amount of a single component that is sufficient to achieve the desired therapeutic result or to have an effect on undesired symptoms, but is generally insufficient to cause adverse side effects.

As used herein, the term “combinatorically effective amount” refers to an amount of multiple components, e.g., an agent that modulates GalR1 and an agent that modulates GalR2, together, that is sufficient to achieve the desired result or to have an effect on an undesired condition. For example, a “combinatorically therapeutically effective amount” refers to an amount of multiple components in total that is sufficient to achieve the desired therapeutic result or to have an effect on undesired symptoms, but is generally insufficient to cause adverse side effects.

As used herein, “patient” or “subject” to be treated includes humans and or non-human animals, including mammals. Mammals include primates, such as humans, chimpanzees, gorillas and monkeys; and domesticated animals.

As used herein, the phrase “EGFR activating mutation(s)” refers to at least one mutation within the protein sequence of EGFR that results in constitutive signaling, which signaling and has been shown to be transforming. Compared to EGFRwt, it is well-known that EGFR activating mutations lead to activation of extensive networks of signal transduction that, in turn, lead to dependence of tumor cells on continuous EGFR signaling for survival.

As used herein, the phrase “EGFR wild type” or EGFRwt refers to epidermal growth factor receptor in its native un-mutated form.

As used herein, the phrase “cancer is resistant to EGFR inhibition” or variations thereof, refers to the well-known mechanism whereby cancer or tumor cells are initially resistant to EGFR inhibition; or have acquired such resistance after initially being susceptible to treatment by a well-known EGFR inhibitor. For example, numerous cancers with activating EGFR mutations, such as non-small cell lung cancers, exhibit a dramatic initial clinical response to treatment with EGFR tyrosine kinase inhibitors (TKIs), but it is well-known that this is followed by the inevitable development of secondary resistance to effective treatment with the particular EGFR inhibitor. As another example well known in the art, resistance to EGFR inhibition can include the emergence of other EGFR mutations such as the T790M mutation that prevent TKI enzyme interaction; as well as activation of other receptor tyrosine kinases such as Met or Axl providing a signaling bypass to EGFR TKI mediated inhibition.

As used herein, a combination refers to any association between two or among more items. The association can be spatial or refer to the use of the two or more items for a common purpose.

As used herein, a pharmaceutical composition refers to any mixture of two or more products or compounds (e.g., agents, modulators, regulators, etc.). It can be a solution, a suspension, liquid, powder, a paste, aqueous or non-aqueous formulations or any combination thereof.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Pharmaceutical Compositions

In one aspect, disclosed are pharmaceutical compositions comprising: (a) an agent that inhibits EGFR signaling, or a pharmaceutically acceptable salt thereof; (b) isoniazid, or a pharmaceutically acceptable salt thereof; and (c) a pharmaceutically acceptable carrier, wherein at least one of the agent that inhibits EGFR signaling and isoniazid is present in an effective amount.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients.

In various aspects, the agent that inhibits EGFR signaling is selected from the group consisting of erlotinib, afatinib, Cetuximab, panitumumab, Erlotinib HCl, Gefitinib, Lapatinib, Neratinib, Lifirafenib, HER2-nhibitor-1, Nazartinib, Naquotinib, Canertinib, Lapatinib, AG-490, CP-724714, Dacomitinib, WZ4002, Sapitinib, CUDC-101, AG-1478, PD153035 HCL, pelitinib, AC480, AEE788, AP26113-analog, OSI-420, WZ3146, WZ8040, AST-1306, Rociletinib, Genisten, Varlitinib, Icotinib, TAK-285, WHI-P154, Daphnetin, PD168393, Tyrphostin9, CNX-2006, AG-18, AZ5104, Osimertinib, CL-387785, Olmutinib, AZD3759, Poziotinib, vandetanib, and necitumumab. In a further aspect, the agent that inhibits EGFR signaling is selected from the group consisting of erlotinib, afatinib, Cetuximab, panitumumab, and Gefitinib. In a still further aspect, the agent that inhibits EGFR signaling is erlotinib.

In a further aspect, the effective amount is a prophylactically effective amount. In a still further aspect, the effective amount is a therapeutically effective amount.

In a further aspect, the effective amount is an individually effective amount of the agent that inhibits EGFR signaling or isoniazid. In a still further aspect, the effective amount is an individually effective amount of the agent that inhibits EGFR signaling. In yet a further aspect, the effective amount is an individually effective amount of isoniazid.

In a further aspect, the effective amount is a combinatorically effective amount of the agent that inhibits EGFR signaling and isoniazid.

Pharmaceutical compositions containing the EGFR inhibitor and the TNF inhibitors, either as separate agents or in combination in a single composition mixture can be formulated in any conventional manner by mixing a selected amount of the respective inhibitor with one or more physiologically acceptable carriers or excipients. Selection of the carrier or excipient is within the skill of the administering profession and can depend upon a number of parameters. These include, for example, the mode of administration (i.e., systemic, oral, nasal, pulmonary, local, topical, or any other mode) and disorder treated. The pharmaceutical compositions provided herein can be formulated for single dosage (direct) administration or for dilution or other modification. The concentrations of the compounds in the formulations are effective for delivery of an amount, upon administration, that is effective for the intended treatment. Typically, the compositions are formulated for single dosage administration. To formulate a composition, the weight fraction of a compound or mixture thereof is dissolved, suspended, dispersed, or otherwise mixed in a selected vehicle at an effective concentration such that the treated condition is relieved or ameliorated.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, Pa. 1990.

Generally, pharmaceutically acceptable compositions are prepared in view of approvals for a regulatory agency or other prepared in accordance with generally recognized pharmacopeia for use in animals and in humans. Pharmaceutical compositions can include carriers such as a diluent, adjuvant, excipient, or vehicle with which an isoform is administered. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions.

It is understood that appropriate doses depend upon a number of factors within the level of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the therapeutic agent to have upon the subject. Exemplary doses include milligram or microgram amounts of the therapeutic agent per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses depend upon the potency. Such appropriate doses may be determined using the assays known in the art. When one or more of these compounds is to be administered to an animal (e.g., a human), a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, and any drug combination.

Parenteral compositions may be formulated in dosage unit form for ease of administration and uniformity of dosage. Dosage unit form as used herein refers to physically discrete units suited as unitary dosages for the subjects to be treated; each unit containing a predetermined quantity of compound of the invention calculated to produce the desired therapeutic effect in association with the required pharmaceutical vehicle. The specification for the dosage unit forms of the invention are dictated by and directly dependent on (a) the unique characteristics of the therapeutic agent and the particular therapeutic effect to be achieved, and (b) the limitations inherent in the art of compounding such a compound of the invention for the treatment of the disease.

### C. Methods of Making a Composition

In one aspect, disclosed are methods for making a disclosed pharmaceutical composition. Thus, in various aspects, disclosed are methods for making a pharmaceutical composition, the method comprising combining: (a) an agent that inhibits EGFR signaling, or a pharmaceutically acceptable salt thereof; (b) isoniazid, or a pharmaceutically acceptable salt thereof; and (c) a pharmaceutically acceptable carrier, wherein at least one of the agent that inhibits EGFR signaling and isoniazid is present in an effective amount.

In a further aspect, the effective amount is a prophylactically effective amount. In a still further aspect, the effective amount is a therapeutically effective amount.

In a further aspect, the effective amount is an individually effective amount of the agent that inhibits EGFR signaling or isoniazid. In a still further aspect, the effective amount is an individually effective amount of the agent that inhibits EGFR signaling. In yet a further aspect, the effective amount is an individually effective amount of isoniazid.

In a further aspect, the effective amount is a combinatorically effective amount of the agent that inhibits EGFR signaling and isoniazid.

In a further aspect, combining is co-formulating the agent that inhibits EGFR signaling and isoniazid with the pharmaceutically acceptable carrier. In a still further aspect, co-formulating provides an oral solid dosage form comprising the agent that inhibits EGFR signaling, isoniazid, and the pharmaceutically acceptable carrier. In yet a further aspect, the solid dosage form is a tablet. In an even further aspect, the solid dosage form is a capsule.

In a further aspect, co-formulating provides an injectable dosage form comprising the agent that inhibits EGFR signaling, isoniazid, and the pharmaceutically acceptable carrier.

### D. Methods of Using the Compositions

Also provided are methods of use of a disclosed composition or medicament. In one aspect, the method of use is directed to the treatment of a disorder. In a further aspect, the disclosed compounds can be used as single agents or in combination with one or more other drugs in the treatment, prevention, control, amelioration, or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

The pharmaceutical compositions and methods of the present invention can further comprise other therapeutically active compounds as noted herein, which are usually applied in the treatment of the above mentioned pathological conditions.

### 1. Treatment Methods

In one aspect, the compounds and compositions disclosed herein are useful for treating, preventing, ameliorating, controlling or reducing the risk of a variety of cancers such as, for example, brain cancer, lung cancer (e.g., non-small cell lung cancer), cervical cancer, ovarian cancer, cancer of the central nervous system (CNS), skin cancer, prostate cancer, sarcoma, breast cancer, leukemia, colorectal cancer, colon cancer, head cancer, neck cancer, endometrial, kidney cancer, and human epithelial carcinoma (e.g., basal cell carcinoma, squamous cell carcinoma, renal cell carcinoma (RCC), ductal carcinoma in situ (DCIS), invasive ductal carcinoma).

The compounds and compositions are further useful in methods for the prevention, treatment, control, amelioration, or reduction of risk of cancers noted herein. The compounds and compositions are further useful in a method for the prevention, treatment, control, amelioration, or reduction of risk of the aforementioned cancers in combination with other agents.

In one aspect, the disclosed compounds can be used in combination with one or more other drugs in the treatment, prevention, control, amelioration, or reduction of risk of cancers for which disclosed compounds or the other drugs can have utility, where the combination of the drugs together are safer or more effective than either drug alone. Such other drug(s) can be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and a disclosed compound is preferred. However, the combination therapy can also include therapies in which a disclosed compound and one or more other drugs are administered on different overlapping schedules. It is also contemplated that when used in combination with one or more other active ingredients, the disclosed compounds and the other active ingredients can be used in lower doses than when each is used singly.

Accordingly, the pharmaceutical compositions can also contain one or more other active ingredients. These combinations include combinations with one other active compound, but also with two or more other active compounds. Likewise, disclosed compositions can be used in combination with other drugs that are used in the prevention, treatment, control, amelioration, or reduction of risk of cancers for which disclosed compounds are useful. Such other drugs can be administered, by a route and in an amount commonly used therefor, contemporaneously or sequentially with a composition of the present invention. When a composition of the present invention is used contemporaneously with one or more other drugs, a pharmaceutical composition containing such other drugs in addition to the disclosed required components is preferred. Accordingly, the pharmaceutical compositions include those that also contain one or more other active ingredients, in addition to the other components disclosed herein.

The weight ratio of a disclosed component to the additional active ingredient can be varied and will depend upon the effective dose of each ingredient. Generally, an effective dose of each will be used. Thus, for example, when a component of the present invention is combined with another agent, the weight ratio of a disclosed component to the other agent will generally range from about 1000:1 to about 1:1000, preferably about 200:1 to about 1:200. Combinations of a component of the present invention and other active ingredients will generally also be within the aforementioned range, but in each case, an effective dose of each active ingredient should be used.

In such combinations, a disclosed composition and other active agents can be administered separately or in conjunction. In addition, the administration of one element can be prior to, concurrent to, or subsequent to the administration of other agent(s).

Accordingly, the subject compositions can be used alone or in combination with other agents which are known to be beneficial in the subject indications or other drugs that affect receptors or enzymes that either increase the efficacy, safety, convenience, or reduce unwanted side effects or toxicity of the disclosed components of the subject compositions. The subject composition and the other agent can be co-administered, either in concomitant therapy or in a fixed combination.

### a. Treating Cancer

In one aspect, disclosed are methods for treating cancer in a patient in need thereof, the method comprising administering to the patient an effective amount of an agent that inhibits EGFR signaling, or a pharmaceutically acceptable salt thereof, and isoniazid (INF), or a pharmaceutically acceptable salt thereof

In various aspects, the agent that inhibits EGFR signaling is selected from the group consisting of erlotinib, afatinib, Cetuximab, panitumumab, Erlotinib HCl, Gefitinib, Lapatinib, Neratinib, Lifirafenib, HER2-nhibitor-1, Nazartinib, Naquotinib, Canertinib, Lapatinib, AG-490, CP-724714, Dacomitinib, WZ4002, Sapitinib, CUDC-101, AG-1478, PD153035 HCL, pelitinib, AC480, AEE788, AP26113-analog, OSI-420, WZ3146, WZ8040, AST-1306, Rociletinib, Genisten, Varlitinib, Icotinib, TAK-285, WHI-P154, Daphnetin, PD168393, Tyrphostin9, CNX-2006, AG-18, AZ5104, Osimertinib, CL-387785, Olmutinib, AZD3759, Poziotinib, vandetanib, and necitumumab. In a further aspect, the agent that inhibits EGFR signaling is selected from the group consisting of erlotinib, afatinib, Cetuximab, panitumumab, and Gefitinib. In a still further aspect, the agent that inhibits EGFR signaling is erlotinib.

In a further aspect, the agent that inhibits EGFR signaling and isoniazid are co-formulated. In a still further aspect, the agent that inhibits EGFR signaling and isoniazid are co-packaged.

In a further aspect, the agent that inhibits EGFR signaling and isoniazid are administered concurrently. In a still further aspect, the agent that inhibits EGFR signaling and isoniazid are not administered concurrently.

In a further aspect, the effective amount is a prophylactically effective amount. In a still further aspect, the effective amount is a therapeutically effective amount.

In a further aspect, the effective amount is an individually effective amount of the agent that inhibits EGFR signaling or isoniazid. In a still further aspect, the effective amount is an individually effective amount of the agent that inhibits EGFR signaling. In yet a further aspect, the effective amount is an individually effective amount of isoniazid.

In a further aspect, the effective amount is a combinatorically effective amount of the agent that inhibits EGFR signaling and isoniazid.

In a further aspect, the patient is a mammal. In a still further aspect, the patient is human.

In a further aspect, the patient has been diagnosed with a need for treatment of cancer prior to the administering step. In a still further aspect, the patient is at risk for developing cancer prior to the administering step.

In a further aspect, the method further comprises identifying a patient in need of treatment of cancer.

In a further aspect, the effective amount is a therapeutically effective amount. In a still further aspect, the effective amount is a prophylactically effective amount.

In a further aspect, the cancer is selected from the group consisting of brain cancer, lung cancer, cervical cancer, ovarian cancer, cancer of the central nervous system (CNS), skin cancer, prostate cancer, sarcoma, breast cancer, leukemia, colorectal cancer, colon cancer, head cancer, neck cancer, endometrial and kidney cancer. In a still further aspect, the cancer is lung cancer. In yet a further aspect, the lung cancer is non-small cell lung cancer.

In a further aspect, the cancer is a human epithelial carcinoma. In a still further aspect, the human epithelial carcinoma is selected from the group consisting of basal cell carcinoma, squamous cell carcinoma, renal cell carcinoma (RCC), ductal carcinoma in situ (DCIS), and invasive ductal carcinoma.

In a further aspect, the cancer expresses EGFR wild type. In a still further aspect, the cancer expresses EGFR that contains at least one EGFR activating mutation. In yet a further aspect, the cancer is resistant to EGFR inhibition.

In accordance with the present invention, it has been demonstrated that a rapid increase in TNF levels is a universal response to inhibition of EGFR signaling in lung cancer cells, regardless of whether EGFR is mutant or wild type; and this rapid increase in TNF levels is even detected in cells expressing the T790M mutation. EGFR normally suppresses TNF levels by induction of miR-21 that negatively regulates TNF mRNA stability. It has now been found that inhibition of EGFR signaling results in decreased miR-21 and a rapid upregulation of TNF. TNF then activates NF-κB, which in turn leads to a further increase in TNF transcription, generating a feedforward loop. The biological effect of this TNF driven adaptive response is tumor cell survival despite cessation of EGFR signaling. Of great clinical translational importance in accordance with the present invention, it has been found that Inhibition of the TNF adaptive response renders previously EGFR TKI resistant EGFRwt tumor cells sensitive to EGFR inhibition, suggesting that such resistant cells are still potentially “oncogene addicted” but protected from EGFR TKI induced cell death by this adaptive response. Biological inhibition of TNF signaling or treatment with the clinically available agents Etanercept (Enbrel® or thalidomide results in lung cancer sensitivity to EGFR TKI's in previously EGFR TKI resistant cells. As noted, NSCLCs with EGFR activating mutations respond clinically to EGFR inhibition despite the well-documented adaptive survival responses such as STAT3 activation triggered in these cells by EGFR inhibition. Similarly, increased TNF secretion in response to EGFR inhibition, fails to completely protect EGFR mutant oncogene addicted cancers. However, TNF inhibition enhances the effectiveness of EGFR inhibition in oncogene addicted lung cancers. Importantly, exogenous TNF also protects oncogene addicted tumor cells from loss of EGFR signaling. Without wishing to be bound by theory, the data herein suggest a key role for TNF signaling in inducing primary resistance to EGFR inhibition in lung cancer.

The epidermal growth factor receptor (EGFR) is widely expressed in lung cancer and represents an important therapeutic target. However, EGFR inhibition using tyrosine kinase inhibitors is effective only in the 10-15 percent of cases that harbor activating EGFR activating mutations. For the remainder of cases—of which the majority express wild type EGFR—EGFR inhibition has minimal efficacy and is no longer an approved therapy. In accordance with the present invention, it has been found that a combined inhibition of EGFR and TNF renders previously EGFR TKI resistant EGFRwt tumor cells sensitive to EGFR inhibition, indicating that such resistant cells are still potentially “oncogene addicted” but protected from EGFR TKI induced cell death by a TNF driven adaptive survival response. Thus, a combined inhibition of EGFR and TNF in accordance with the present invention is believed to greatly expand the reach and impact of EGFR targeted treatment in NSCLC.

An important finding provided herein is the identification of an early and widespread mechanism that mediates primary resistance to EGFR inhibition in lung cancer cells, regardless of whether EGFR is wild type or mutant. NSCLC cells respond to EGFR inhibition with a rapid increase in TNF levels and the TNF upregulation was detected in all NSCLC cell lines examined, in animal tumors derived from NSCLC cell lines, and in a direct xenograft model. In the case of EGFR wild type expressing NSCLCs the increase in TNF appears sufficient to protect cells from loss of EGFR signaling. Since the majority of NSCLC express EGFR, this adaptive mechanism is likely triggered in the majority of NSCLC treated with EGFR inhibition. The TNF driven adaptive response is also detected in lung cancer cells with EGFR activating mutations and seemingly conflicts with the proven initial effectiveness of EGFR inhibition in such patients. This is likely because the EGFR activating mutations in oncogene addicted cells lead to activation of extensive signaling networks resulting in an exquisite reliance on EGFR signaling. Thus, the TNF upregulation triggered by EGFR inhibition in these cells is only partially protective and the protection is detected only at low concentrations of EGFR inhibitors. STAT3 is also rapidly activated upon EGFR inhibition in NSCLCs with EGFR activating mutations and does not seem to inhibit the clinical response in patients. Thus, EGFR inhibited in oncogene addicted cells in the clinical setting may trigger adaptive responses that are ineffective or partially effective. Interestingly, a biologically significant TNF upregulation can also be detected in cells harboring the T790M mutation. The T790M mutation is a frequent mechanism for secondary resistance in tumors that are initially sensitive to EGFR inhibition. Thus, the upregulation of TNF in response to EGFR inhibition appears to be a universal feature of EGFR expressing NSCLCs. The upregulation of TNF in the animal models disclosed herein is rapid and peaks around 2-7 days, receding in 7-14 days which makes it difficult to document the TNF upregulation in archival patient tumor specimens, since tissue is rarely resampled at such early times after EGFR inhibition.

EGFR expression is common in NSCLC and intermediate or high levels of EGFR have been detected in 57 to 62% of NSCLCs by immunohistochemistry. EGFR mutations are detected in 10-15% of patients in Caucasians and are found in a higher percentage of Asian populations. The clinical response to EGFR inhibition in tumors with EGFR activating mutations illustrates both the promise and the difficulties of targeted treatment. It became apparent that patients who clearly responded to EGFR inhibition inevitably developed a secondary resistance to this treatment. Thus, overcoming mechanisms of resistance to targeted treatment is critical to the success of targeted treatment and some insights have emerged into mechanisms of secondary resistance to EGFR inhibition in lung cancer. The emergence of secondary resistance implies the persistence of subsets of cancer cells that are not eliminated during the initial exposure of cells to targeted treatment. Thus, a more effective elimination of cancer cells during the initial exposure to targeted treatment may delay or abrogate the emergence of secondary resistance. In addition, it may be possible to overcome the secondary resistance of human epithelial cancers, such as NSCLC and the like, with appropriately targeted treatments such as the methods provided herein.

Primary or intrinsic resistance to EGFRwt inhibition could occur because the EGFRwt does not drive the survival/proliferation of these cells. The alternative possibility is that an adaptive response prevents cell death in response to EGFR inhibition. Currently, most of the attention is focused on the subset of cancers with EGFR activating mutations and the general assumption may be that EGFRwt is not a useful target for treatment, because, although EGFRwt expression is common, EGFR inhibition is ineffective in EGFRwt expressing NSCLC. Furthermore, EGFR mutants are constitutively active and more oncogenic compared to EGFRwt, and engage more signaling networks in cancer cells resulting in a state of dependence or oncogene addiction in EGFR mutant expressing cells. However, the presence of EGFR ligand is common and well documented in lung cancer. Furthermore, a constitutive overexpression induced EGFRwt signaling has also been reported. Thus, it seems likely that EGFRwt expressing cells are also activated in lung cancer. The data provided herein indicate that EGFRwt expressing lung cancer cells can also be rendered sensitive to EGFR inhibition if the TNF adaptive response is inhibited. This finding, in combination with the therapeutic methods provided herein, is believed to broaden the use of EGFR inhibition as an effective treatment in epithelial cancers, such as lung cancer, to include EGFRwt expressing cancers if combined with a TNF inhibitor.

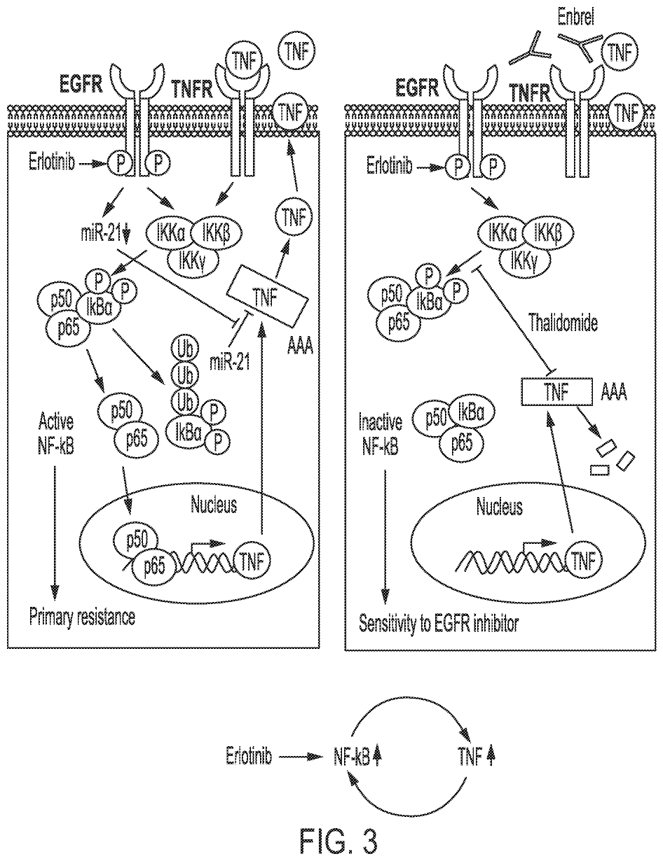

It is contemplated that EGFR inhibition results in an increase in TNF levels via a dual mechanism (as shown in the schematic in FIG. 3). First, it has been demonstrated that activation of EGFR signaling results in a rapid downregulation of TNF mRNA. This temporal profile suggests an effect on RNA stability. Indeed, it has been found that inhibition of EGFR results in increased TNF mRNA stability. It is contemplated that EGFR signaling actively suppresses TNF Levels by inducing specific microRNAs that inhibit TNF mRNA stability. MiR-21 was identified as a plausible candidate, because it is both rapidly induced by EGFR signaling in lung cancer cells and also reported to negatively regulate TNF mRNA. It has been confirmed that miR-21 is rapidly upregulated in lung cancer cell lines when EGFR is activated and also that inhibition of miR-21 inhibits EGFR induced TNF upregulation. A second mechanism that also operates early involves the transcription factor NF-κB. TNF activates NF-κB, which in turn, increases the transcription of TNF mRNA in a feedforward loop. Inhibition of NF-κB also blocks the erlotinib-induced upregulation of TNF levels. In addition, inhibition of TNFR1 also blocks erlotinib-induced upregulation of TNF, confirming the existence of a feed forward loop. The TNF-mediated activation of NF-κB is likely to be a major mechanism of resistance to EGFR inhibition.

The biological effect of increased TNF signaling is protection from cell death mediated by a loss of EGFR signaling. When the TNF mediated adaptive response is blocked, there is an enhanced sensitivity to EGFR inhibition. Conversely, exogenous TNF protects lung cancer cells with EGFR activating mutations from cell death resulting from EGFR inhibition. Inhibition of TNF signaling in sensitive cells with EGFR activating mutations results in an increased sensitivity to EGFR inhibition. Surprisingly, it has been found that TNF inhibition results in rendering EGFRwt expressing cells sensitive to EGFR inhibition. The combined effect of TNF and EGFR inhibition in a resistant EGFRwt cell line A549 cells was examined in a mouse model using multiple approaches to inhibit TNF.

A combination of EGFR TKI plus thalidomide was highly effective in inhibiting tumor growth, while EGFR inhibition or thalidomide alone was ineffective. Thalidomide is a known inhibitor of TNF and may regulate TNF transcription and/or stability. A substantial reduction in tumor growth was also noted in A549 cells with stably silencing of TNF, and with Etanercept, a specific inhibitor of TNF signaling, with a greater than 50% reduction of tumor growth, while inhibition of TNF alone had no significant effect. Using a low concentration of erlotinib, a significant reduction was noted in tumor growth with a combined inhibition of TNF and EGFR using the oncogene addicted cell line, HCC827 cells compared to EGFR inhibition alone, although the tumors were sensitive to EGFR inhibition alone. Thalidomide alone had no effect. A biologically significant upregulation of TNF upon EGFR inhibition may have enormous implications for the treatment of lung cancer. Lung cancer is the most common cancer worldwide, with NSCLC comprising about 85% of all lung cancer. A majority of NSCLC express EGFRwt with a smaller subset expressing EGFR activating mutations. The therapeutic approach provided herein is applicable to the majority of NSCLC including EGFRwt expressing cancers, and include the subset with EGFR activating mutations. In accordance with the present invention, it is believed that inhibiting the EGFR with a combination of TKI plus a TNF inhibitor such as thalidomide or Enbrel is effective in the treatment of human epithelial cancers, such as NSCLCs, and the like, that express EGFRwt. In the subset of tumors with EGFR activating mutations, a combined treatment with EGFR and TNF inhibition is believed to result in a more effective elimination of tumor cells during the initial treatment and perhaps eliminate or delay secondary resistance. A number of TNF inhibiting drugs and antibodies are safe and currently in use in various rheumatologic and immune diseases, making it easy to test this approach in patients. TNF upregulation has also been found in H1975 cells, which harbor a T790M mutation, and it has been found that combined TNF and EGFR inhibition overcomes resistance to EGFR inhibition in these cells, indicating that this approach can be effective in tumors with secondary resistance. EGFR expression is widespread in other types of human cancer, and it is contemplated herein that a biologically significant upregulation of TNF in response to EGFR inhibition is widespread feature of human epithelial cancer, such that the invention methods and compositions provided herein will be effective for treating human epithelial cancers generally.

### 2. Manufacture of a Medicament

In one aspect, the invention relates to a medicament comprising one or more agents that inhibit EGFR signaling, or a pharmaceutically acceptable salt thereof, and isoniazid, or a pharmaceutically acceptable salt thereof.

In various aspect, the invention relates methods for the manufacture of a medicament for treating cancer comprising combining one or more disclosed compounds, products, or compositions or a pharmaceutically acceptable salt thereof, with a pharmaceutically acceptable carrier. It is understood that the disclosed methods can be performed with the disclosed compounds, products, and pharmaceutical compositions. It is also understood that the disclosed methods can be employed in connection with the disclosed methods of using.

### 3. Use of Compounds and Compositions

Also provided are the uses of the disclosed compounds and compositions. Thus, in one aspect, disclosed are uses of at least one agent that inhibits EGFR signaling, or a pharmaceutically acceptable salt thereof, and isoniazid, or a pharmaceutically acceptable salt thereof.

In a further aspect, disclosed are uses of at least one agent that inhibits EGFR signaling, or a pharmaceutically acceptable salt thereof, and isoniazid, or a pharmaceutically acceptable salt thereof, in the manufacture of a medicament for the treatment of a cancer.

In a further aspect, the use relates to a process for preparing a pharmaceutical composition comprising a therapeutically effective amount of at least one agent that inhibits EGFR signaling, or a pharmaceutically acceptable salt thereof, and isoniazid, or a pharmaceutically acceptable salt thereof, for use as a medicament.

In a further aspect, the use relates to a process for preparing a pharmaceutical composition comprising a therapeutically effective amount of at least one agent that inhibits EGFR signaling, or a pharmaceutically acceptable salt thereof, and isoniazid, or a pharmaceutically acceptable salt thereof, wherein a pharmaceutically acceptable carrier is intimately mixed with a therapeutically effective amount of the at least one agent that inhibits EGFR signaling and/or isoniazid.

In various aspects, the use relates to the treatment of a cancer in a vertebrate animal. In a further aspect, the use relates to the treatment of a cancer in a human subject.

In a further aspect, the use is the treatment of a cancer. In a still further aspect, the cancer is brain cancer, lung cancer, cervical cancer, ovarian cancer, cancer of the central nervous system (CNS), skin cancer, prostate cancer, sarcoma, breast cancer, leukemia, colorectal cancer, colon cancer, head cancer, neck cancer, endometrial and kidney cancer. In a still further aspect, the cancer is lung cancer. In yet a further aspect, the lung cancer is non-small cell lung cancer.

In a further aspect, the cancer is a human epithelial carcinoma. In a still further aspect, the human epithelial carcinoma is selected from the group consisting of basal cell carcinoma, squamous cell carcinoma, renal cell carcinoma (RCC), ductal carcinoma in situ (DCIS), and invasive ductal carcinoma.

In a further aspect, the cancer expresses EGFR wild type. In a still further aspect, the cancer expresses EGFR that contains at least one EGFR activating mutation. In yet a further aspect, the cancer is resistant to EGFR inhibition.

It is understood that the disclosed uses can be employed in connection with the disclosed compounds, methods, compositions, and kits. In a further aspect, disclosed are uses of a disclosed compound or composition of a medicament for the treatment of a cancer in a mammal.

In a further aspect, disclosed are uses of a disclosed compound or composition in the manufacture of a medicament for the treatment of a cancer selected from brain cancer, lung cancer, cervical cancer, ovarian cancer, cancer of the central nervous system (CNS), skin cancer, prostate cancer, sarcoma, breast cancer, leukemia, colorectal cancer, colon cancer, head cancer, neck cancer, endometrial and kidney cancer. In a still further aspect, the cancer is lung cancer such as, for example, non-small cell lung cancer. In a still further aspect, the cancer is human epithelial carcinoma such as, for example, basal cell carcinoma, squamous cell carcinoma, renal cell carcinoma (RCC), ductal carcinoma in situ (DCIS), and invasive ductal carcinoma.

In a further aspect, disclosed are uses of a disclosed compound or composition in the manufacture of a medicament for the treatment of a cancer.

In various aspects, the agents and methods described herein can be used prophylactically, such as to prevent, reduce or delay progression of a cancer.

### 4. Kits

In one aspect, disclosed are kits comprising an agent that inhibits EGFR signaling, or a pharmaceutically acceptable salt thereof, and isoniazid, or a pharmaceutically acceptable salt thereof, and one or more of: (a) an agent associated with the treatment of cancer; (b) instructions for administering the agent that inhibits EGFR signaling and/or isoniazid in connection with treating cancer; and (c) instructions for treating cancer.

In a further aspect, the agent that inhibits EGFR signaling and isoniazid are co-formulated. In a still further aspect, the agent that inhibits EGFR signaling and isoniazid are co-packaged.

In a further aspect, the agent is a chemotherapeutic agent. Examples of chemotherapeutic agent include, but are not limited to, alkylating agents, antimetabolite agents, antineoplastic antibiotic agents, mitotic inhibitor agents, and mTor inhibitor agents.

In a further aspect, the chemotherapeutic agent is an alkylating agent. Examples of alkylating agents include, but are not limited to, carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof

In a further aspect, the chemotherapeutic agent is an antimetabolite agent. Examples of antimetabolite agents include, but are not limited to, gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

In a further aspect, the chemotherapeutic agent is an antineoplastic antibiotic agent. Examples of antineoplastic antibiotic agents include, but are not limited to, doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

In a further aspect, the chemotherapeutic agent is a mitotic inhibitor agent. Examples of mitotic inhibitor agents include, but are not limited to, irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof.

In a further aspect, the chemotherapeutic agent is an mTOR inhibitor agent. Examples of mTOR inhibitor agents include, but are not limited to, everolimus, siroliumus, and temsirolimus, or a pharmaceutically acceptable salt, hydrate, solvate, or polymorph thereof.

In a further aspect, the agent that inhibits EGFR signaling, isoniazid, and the chemotherapeutic agent are co-packaged. In a still further aspect, the agent that inhibits EGFR signaling, isoniazid, and the chemotherapeutic agent are administered sequentially. In yet a further aspect, the agent that inhibits EGFR signaling, isoniziad, and the chemotherapeutic agent are administered simultaneously.

In various aspects, the agents and pharmaceutical compositions described herein can be provided in a kit. The kit can also include combinations of the agents and pharmaceutical compositions described herein. The kit can include: a) one or more agents, such as in a composition that includes the agents; b) informational material; and any combination of a) and b). The informational material can be descriptive, instructional, marketing or other material that relates to the methods described herein and/or to the use of the agents for the methods described herein. For example, the informational material relates to the use of the agents herein to treat a subject who has, or who is at risk for developing, a cancer.

In various aspects, the informational material can include instructions for administering the pharmaceutical composition and/or cell(s) in a suitable manner to treat a human, e.g., in a suitable dose, dosage form, or mode of administration (e.g., a dose, dosage form, or mode of administration described herein). In a further aspect, the informational material can include instructions to administer the pharmaceutical composition to a suitable subject, e.g., a human having, or at risk for developing, cancer.

In various aspects, the composition of the kit can include other ingredients, such as a solvent or buffer, a stabilizer, a preservative, a fragrance or other cosmetic ingredient. In such aspects, the kit can include instructions for admixing the agent and the other ingredients, or for using one or more compounds together with the other ingredients.

In a further aspect, the agent that inhibits EGFR signaling and isoniazid are co-formulated. In a still further aspect, the agent that inhibits EGFR signaling and isoniazid are co-packaged.

In a further aspect, the kit further comprises a plurality of dosage forms, the plurality comprising one or more doses; wherein each dose comprises the agent that inhibits EGFR signaling, and the chemotherapeutic agent, wherein at least one is present in an effective amount. In a still further aspect, the effective amount is a therapeutically effective amount. In yet a further aspect, the effective amount is a prophylactically effective amount. In an even further aspect, each dose of the agent that inhibits EGFR signaling and the chemotherapeutic agent are co-packaged. In a still further aspect, each dose of the agent that inhibits EGFR signaling and the chemotherapeutic agent are co-formulated.

In a further aspect, the kit further comprises a plurality of dosage forms, the plurality comprising one or more doses; wherein each dose comprises isoniazid, and the chemotherapeutic agent, wherein at least one is present in an effective amount. In a still further aspect, the effective amount is a therapeutically effective amount. In yet a further aspect, the effective amount is a prophylactically effective amount. In an even further aspect, each dose of isoniazid and the chemotherapeutic agent are co-packaged. In a still further aspect, each dose of isoniazid and the chemotherapeutic agent are co-formulated.

In a further aspect, the dosage forms are formulated for oral administration. In a still further aspect, the dosage forms are formulated for intravenous administration.

### 5. Subjects

In various aspects, the subject of the herein disclosed methods is a vertebrate, e.g., a mammal. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

In some aspects of the disclosed methods, the subject has been diagnosed with a need for treatment prior to the administering step. In some aspects of the disclosed method, the subject has been diagnosed with cancer prior to the administering step. In some aspects of the disclosed methods, the subject has been identified with a need for treatment prior to the administering step. In one aspect, a subject can be treated prophylactically with a compound or composition disclosed herein, as discussed herein elsewhere.

### a. Dosage

Toxicity and therapeutic efficacy of the agents and pharmaceutical compositions described herein can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and can be expressed as the ratio LD₅₀/ED₅₀. Polypeptides or other compounds that exhibit large therapeutic indices are preferred.

Data obtained from cell culture assays and further animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any agents used in the methods described herein, the therapeutically effective dose can be estimated initially from cell culture assays. A dose can be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Exemplary dosage amounts of a differentiation agent are at least from about 0.01 to 3000 mg per day, e.g., at least about 0.00001, 0.0001, 0.001, 0.01, 0.1, 1, 2, 5, 10, 25, 50, 100, 200, 500, 1000, 2000, or 3000 mg per kg per day, or more.

The formulations and routes of administration can be tailored to the disease or disorder being treated, and for the specific human being treated. For example, a subject can receive a dose of the agent once or twice or more daily for one week, one month, six months, one year, or more. The treatment can continue indefinitely, such as throughout the lifetime of the human. Treatment can be administered at regular or irregular intervals (once every other day or twice per week), and the dosage and timing of the administration can be adjusted throughout the course of the treatment. The dosage can remain constant over the course of the treatment regimen, or it can be decreased or increased over the course of the treatment.

In various aspects, the dosage facilitates an intended purpose for both prophylaxis and treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., (1990) Chapter 27 in Remington's Pharmaceutical Sciences, 18th Ed., Gennaro, ed., Mack Publishing Co., Easton, Pa.). In general, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., (1996) Chapter 3, In: Goodman & Gilman's The Pharmacological Basis of Therapeutics, 9th Ed., Hardman et al., eds., McGraw-Hill, New York, N.Y.).

### b. Routes of Administration

Also provided are routes of administering the disclosed compounds and compositions. The compounds and compositions of the present invention can be administered by direct therapy using systemic administration and/or local administration. In various aspects, the route of administration can be determined by a patient's health care provider or clinician, for example following an evaluation of the patient. In various aspects, an individual patient's therapy may be customized, e.g., the type of agent used, the routes of administration, and the frequency of administration can be personalized. Alternatively, therapy may be performed using a standard course of treatment, e.g., using pre-selected agents and pre-selected routes of administration and frequency of administration.

Systemic routes of administration can include, but are not limited to, parenteral routes of administration, e.g., intravenous injection, intramuscular injection, and intraperitoneal injection; enteral routes of administration e.g., administration by the oral route, lozenges, compressed tablets, pills, tablets, capsules, drops (e.g., ear drops), syrups, suspensions and emulsions; rectal administration, e.g., a rectal suppository or enema; a vaginal suppository; a urethral suppository; transdermal routes of administration; and inhalation (e.g., nasal sprays).

In various aspects, the modes of administration described above may be combined in any order. In this respect, one or more agents that inhibit EGFR signaling can be administered before, after, or simultaneously with isoniazid.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter, which is broadly contemplated by the present invention. It is further understood that the scope of the present invention fully encompasses other embodiments that may become obvious to those skilled in the art and that the scope of the present invention is accordingly not limited.

### E. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

In addition, the following Materials and Methods are also as described in WO 2018/075823 A1, which is incorporated herein by reference in its entirety.

### 1. Materials and Methods

### a. Plasmids, Transfection, and Generation of Cell Lines

Calu-3 and A549 cells were obtained from ATCC. All other cell lines were obtained from the Hamon Center for Therapeutic Oncology Research at the University of Texas Southwestern Medical Center (and deposited at the ATCC). Cells were cultured in RPMI-1640 in 5% FBS for all experiments except for experiments involving the use of EGF. Cell lines were DNA fingerprinted using Promega StemElite ID system, which is an STR based assay at UT Southwestern genomics core and mycoplasma tested using an e-Myco kit (Boca Scientific). p65 expression plasmid was obtained from Stratagene (La Jolla, Calif.). NF-κB-LUC plasmid was provided by Dr. Ezra Burstein (UT Southwestern). At least 3 independent experiments were performed unless otherwise indicated.

### b. Luciferase Assays

Cells were plated in 48 well dishes followed by transfection with NF-κB-LUC plasmid using lipofectamine 2000. A dual-luciferase reporter assay system was used according to the instructions of the manufacturer (Promega, Madison Wis.). Firefly luciferase activity was measured in a luminometer and normalized on the basis of Renilla luciferase activity. Experiments were done in triplicate and 3 independent experiments were done.

### c. RNA Interference

For transient silencing, a pool was used of siRNA sequences directed against human TNFR1 or control (scrambled) siRNA all obtained from Santa Cruz Biotechnology (Dallas, Tex.). siRNA knockdown was performed according to the manufacturer's protocol using Lipofectamine 2000 reagent (Invitrogen Carlsbad, Calif.). Experiments were conducted 48h after siRNA transfection.

### d. Antibodies, Reagents and Western Blotting

Western blot and immunoprecipitation were performed according to standard protocols. In all experiments involving use of EGF, cells were cultured overnight in serum free RPMI-1640 and EGF was added to serum free medium. In such experiments, cells not treated with EGF were also serum starved. Erlotinib was purchased from SelleckChem (Houston, Tex.). pEGFR (2236), pERK (4376), ERK (4695), pJNK (9251), JNK (9252), NF-κB p65 (8242), IκBα (4814) antibodies were from Cell Signaling Technology (Danvers, Mass.); TNFR1 (sc-8436), and β-Actin (sc-47778) were from Santa Cruz Biotechnology (Dallas, Tex.); EGFR (06-847) was from EMD Millipore (Billerica, Mass.).

Reagents: Recombinant human TNF and EGF was obtained from Peprotech (Rocky Hill, N.J.). Erlotinib was purchased from SelleckChem (Houston, Tex.). Afatinib was bought from AstaTech, Inc. (Bristol, Pa.). Thalidomide and Mithramycin (MMA) were from Cayman Chemical (Ann Arbor, Mich.). Enbrel (Etanercept) was purchased from Mckesson Medical Supply (San Francisco Calif.). The NF-κB inhibitors, BMS-345541, QNZ (EVP4593), and sodium salicylate were obtained from EMD Millipore (Billerica, Mass.).

### e. Chromatin Immunoprecipitation Assay

HCC827, H3255, H441, or A549 cells were plated in 15 cm plates per reaction for ChIP assay (2×106 cells). The ChIP assay was carried out by using Chromatin Immunoprecipitation (ChIP) Assay Kit (Millipore) according to standard protocols (Nelson et al., 2006). For qPCR 2 μl of DNA from each reaction was mixed with SYBR Green Master Mix (Applied Biosystems, CA) and carried out in ViiA 7 Real-Time PCR System (Applied Biosystems). The data are expressed as percentage of input. Putative NF-κB binding sites on TNF promoter were predicted by running AliBaba 2.1 program, and two sites were examined. The following 2 primer pairs were used: Region 1 (-1909/-1636) covering putative NF-κB binding site (-1812/-1801): (SEQ ID NO:1) 5′-CCGGAGCTTTCAAAGAAGGAATTCT-3′ (forward) and (SEQ ID NO:2) 5′-CCCCTCTCTCCATCCTCCATAAA-3′ (reverse); Region 2 (-1559/-1241) covering putative NF-κB binding site (-1513/-1503): (SEQ ID NO:3) 5′-ACCAAGAGAGAAAGAAGTAGGCATG-3′ (forward) and (SEQ ID NO:4) 5′-AGCAGTCTGGCGGCCTCACCTGG-3′ (reverse).

### f. cDNA Synthesis and Real Time PCR

Total RNA was isolated by TRIzol Reagent (Ambion). cDNA Reverse Transcription was performed by using High-Capacity cDNA Reverse Transcription Kit (Applied Biosystems). PCR primers were synthesized by IDT (Coralville, Iowa). Each PCR was carried out in triplicate in a 20 μl volume using SYBR Green Master Mix (Applied Biosystems) for 15 minutes at 95° C. for initial denaturing, followed by 40 cycles of 95° C. for 15 s and 60° C. for 60 s in ViiA 7 Real-Time PCR System (Applied Biosystems). At least three independent experiments were done. Values for each gene were normalized to expression levels of GAPDH mRNA. Primer sequences were as below. TNF: (SEQ ID NO:5) 5′-CCCAGGGACCTCTCTCTAATCA-3′ (forward) and (SEQ ID NO:6) 5′-GCTACAGGCTTGTCACTCGG-3′ (reverse); GAPDH: (SEQ ID NO:7) 5′-GTGAAGGTCGGAGTCAACGG-3′ (forward) and (SEQ ID NO:8) 5′-TGATGACAAGCTTCCCGTTCTC-3′ (reverse).

### g. MicroRNA Studies

For microRNA quantitation, mirVana miRNA Isolation Kit (Ambion) was used to isolate the high-quality small RNAs. TaqMan MicroRNA Reverse Transcription Kit (Applied Biosystems) was used for converting miRNA to cDNA. The RT primers were within the Taqman MicroRNA Assay hsa-miR-21-5p and hsa-miR-423-5p (ThermoFisher). hsa-miR-423-5p was used as the endogenous control. PCR reactions were performed in triplicate by TaqMan® Universal Master Mix II (Applied Biosystems), using the same PCR program as SYBR Green Master Mix. PCR primers of hsa-miR-21-5p and hsa-miR-423-5p were from Taqman MicroRNA Assay (ThermoFisher). Each experiment was carried out independently at least twice. The miR-21 expression levels were normalized to miR-423.

For microRNA inhibition, miRNA inhibitors were obtained from IDT (Coralville, Iowa). The mature sequence of hsa-miR-21-5p was achieved from www.mirbase.org as (SEQ ID NO:9) uagcuuaucagacugauguuga; The human negative control miRNA inihibitor sequence was proposed by IDT as (SEQ ID NO:10) ucguuaaucggcuauaauacgc. miRNA inhibitors were transfected into cultured cells by a method similar to siRNA transfection, using Lipofectamine 2000 reagent.

### h. ELISA

To detect TNF levels in medium, cells were cultured in serum free medium and treated with indicated drugs for 48 hours. Supernatant was then collected and concentrated using a Pierce protein concentrator (Thermo-Fisher). To test TNF in lysates, cell and tumor lysates were extracted following standard protocols used for Western blot. Total protein concentrations were determined by Pierce BCA Protein Assay Kit (Fisher Scientific). Then, the levels of TNF protein were measured by ELISA using a commercial TNF detection kit (Fisher Scientific) according to the manufacturer's instruction.

### i. Virus Infection

Adenovirus-GFP or IkBα adenovirus were obtained from Vector Biolabs (Malvern, Pa.). An MOI of 10 was used in the experiments. Cells were exposed to adenovirus in the presence or absence of Erlotinib for 72 h followed by Cell viability assay or Western blotting.

Human shTNF Lentiviral Particles and Control shRNA Lentiviral Particles-A were purchased from Santa Cruz Biotechnology (Dallas, Tex.). Cells were infected with shRNA lentiviral particles following the manufacturer's protocol and 0.6 μg/mL puromycin was added for selecting stable clones.

### j. Cell Viability Assay

Cell viability assay was conducted using AlamarBlue cell viability assay from Thermo-Fisher, according to the manufacturer's protocol. Cells were treated by indicated drugs for 72 h before detection. In AlamarBlue cell viability assay, cells were cultured at Corning 96-well black plates with clear bottom, and the detection was carried out under the fluorimeter (excitation at 544 nm and emission at 590 nm) using POLARstar Omega Microplate Reader (BMG LABTECH, Germany).

### k. Animal Studies

4 to 6 weeks old female athymic mice were purchased from Charles River Laboratories. 1×106 A549 or 2×106 HCC827 cells were subcutaneously injected into the flanks of athymic mice. After about 10 days post injection, all mice had developed subcutaneous tumors. The mice were randomly divided into control and treatment groups, mice were treated with drugs using the doses described in the figure legends of WO 2018/075823A1 (which is incorporated herein by reference in its entirety) for 10 days. For combination treatment, both drugs were given concurrently for indicated periods. Tumor dimensions were measured every two days and tumor volumes calculated by the formula: volume=length×length×width/2. Mice were sacrificed when tumors reached over 2000 m³ or after 24 days.

HCC4087 PDX model was established at UT Southwestern. The NSCLC specimen (P0) was surgically resected from a patient diagnosed with adenocarcinoma/squamous cell carcinoma, IIB, T3, at UT Southwestern, after obtaining Institutional Review Board approval and informed consent. It has KRAS G13C mutation but no EGFR activating mutations in the normal lung or lung tumor detected by Exome sequencing. 4 to 6 weeks old female NOD SCID mice were purchased from Charles River Laboratories. The PDX tumor tissues were cut into small pieces (˜20 mm³) and subcutaneously implanted in NOD SCID mice of serial generations (P1, P2, etc.). P4 tumor bearing SCID mice were used in this study.

All animal studies were done under Institutional Animal Care and Use Committee-approved protocols at the University of Texas Southwestern Medical Center and North Texas VA Medical Center.

### 1. Statistical Analysis

Error bars represent the means±SEM of three independent experiments. All data were analyzed for significance with Student's t-test using GraphPad Prism 7.0 software, where P<0.05 was considered statistically significant. * means that P<0.05, ** means that P<0.01, and *** indicates any p value less than 0.001. # indicates not statistically significant.

### 2. Results

### a. EGFR Inhibition Leads to Upregulation of TNF Expression in Lung Cancer Cell Lines and Xenograft Tumors

Previous studies have shown that exposure of lung cancer cells to EGFR tyrosine kinase inhibitors such as erlotinib results in a rapid activation of NF-κB in EGFR mutant NSCLC cells. The activation of NF-κB is biologically significant and appears to protect cancer cells from cell death resulting from EGFR inhibition. TNF is a key activator of NF-κB, and the possibility that TNF may mediate the NF-κB activation triggered by EGFR inhibition was evaluated. First, whether erlotinib induced an increase in TNF levels in lung cancer cell lines was investigated. Without wishing to be bound by theory, it was found that exposure of lung cancer cell lines to erlotinib resulted in increased TNF mRNA levels in all 18 cell lines examined as determined by real time quantitative PCR. The cell lines used in this study with EGFR mutation status are listed in Table 1 below.

TABLE 1

| | Cell Lines | EGFR Status |
|---|---|---|
| 1 | H3255 | Mutant(L858R) |
| 2 | PC9 | Mutant(ex19del) |
| 3 | HCC827 | Mutant(ex19del) |
| 4 | HCC4006 | Mutant(ex19del) |
| 5 | H1373 | Wild type |
| 6 | H1975 | Mutant(L858R/T790M) |
| 7 | H1650 | Mutant(ex19del) |
| 8 | H322 | Wild type |
| 9 | H441 | Wild type |
| 10 | H1666 | Wild type |
| 11 | A549 | Wild type |
| 12 | Calu-3 | Wild type |
| 13 | HCC2279 | Mutant(ex19del) |
| 14 | HCC4011 | Mutant(L858R) |
| 15 | HCC820 | Mutant(ex19del/T790M) |
| 16 | HCC2935 | Mutant(ex19del) |
| 17 | H1573 | Wild type |
| 18 | H2122 | Wild type |

Remarkably, while the temporal profiles vary, the increase in TNF is detected in both EGFRwt and EGFR mutant cell lines. The increase in TNF levels upon EGFR inhibition was confirmed at a protein level by ELISA. A similar result was found with afatinib, an irreversible EGFR inhibitor in various cell lines. Afatinib also induced upregulation of TNF in a resistant cell line H1975 that harbors the EGFR T790M mutation rendering it resistant to first generation TKIs like erlotinib.

Erlotinib also induced upregulation of TNF in tumors growing in mice. Athymic mice were inoculated with EGFR mutant HCC827 or EGFRwt NSCLC A549 cells. Following formation of subcutaneous tumors, mice were treated with erlotinib for various time points. This was followed by removal of tumors. TNF is increased in tumors generated with either EGFRwt expressing lung cancer cell line A549 or EGFR mutant expressing lung cancer cell lines (HCC827) upon treatment with erlotinib. Importantly, increased TNF was also detected in a NSCLC PDX derived from EGFR expressing NSCLC (HCC 4087) without EGFR activating mutations, growing in NOD-SCID mice and treated with erlotinib for the indicated time points.

### b. EGFR Activation Leads to Decrease in TNF mRNA

The increase in TNF mRNA following EGFR inhibition suggests that the EGFR is either actively suppressing TNF levels, or the rise in TNF could be secondary to a feedback mechanism. To examine a direct suppression, cells were treated with EGF to activate the EGFR and the TNF mRNA level was determined. EGF-mediated activation of the EGFR results in a rapid decrease in TNF mRNA levels in both EGFR mutant as well as EGFRwt cell lines. This decrease in TNF mRNA can be detected as early as 15 minutes after EGF exposure, suggesting an effect on TNF mRNA stability rather than transcription. This finding would suggest that EGFR signaling normally keeps the TNF level low and a loss of EGFR signaling results in increased TNF. The EGFR-induced decrease in TNF at a protein level was confirmed by ELISA. Next, whether EGFR activity influences TNF mRNA stability was examined using Actinomycin D as an inhibitor of transcription. It was found that inhibition of the EGFR with erlotinib leads to an increase in TNF mRNA stability.

### c. EGFR Regulates TNF mRNA via Expression of MicroRNA-21

MicroRNAs represent an important and rapidly inducible mechanism of regulating mRNA stability and translation. Previous studies have demonstrated that EGFR regulates the expression of specific miRNAs in lung cancer cells. Importantly, studies have shown that EGFR regulates miRNA levels in lung cancer. Without wishing to be bound by theory, it was hypothesized that EGFR activity may regulate TNF mRNA stability by a mechanism involving expression of specific miRNA. Previous studies have also reported that miR-21, one of the microRNAs that is regulated by EGFR activity in lung cancer cells, is also known to negatively regulate TNF mRNA levels. Thus, microRNA mediated regulation of TNF mRNA seemed like a plausible mechanism of rapid regulation of TNF mRNA stability by EGFR signaling. The upregulation of miR-21 by EGFR activity and its downregulation by EGFR inhibition was initially confirmed in multiple lung cancer cell lines. Next, the effect of antisense miR-21 on EGFR-induced downregulation of TNF was examined. Indeed, it was found that inhibition of miR-21 results in a rescue of EGF-induced downregulation of TNF in multiple EGFR mutant and EGFRwt cell lines. miR-21 inhibition was confirmed by real time quantitative PCR.

### d. Erlotinib Induced NF-κB Activation is Mediated by TNF

Next, whether the increased TNF plays a role in erlotinib-induced NF-κB activation was examined. A recent study has reported that NF-κB is rapidly activated in lung cancer cells expressing EGFR activating mutations. It was confirmed that NF-κB was activated by erlotinib in EGFR mutant cell lines and found that NF-κB is also activated in cell lines that express EGFRwt using a reporter assay. NF-κB activation was also confirmed by degradation of IKBα following erlotinib treatment. Thus, the activation of NF-κB is seen in both EGFRwt as well as EGFR mutant expressing cell lines. Since TNF is a major activator of NF-κB, the possibility that erlotinib activated NF-κB via an increase in TNF level was considered. TNFR1 is expressed widely, while TNFR2 expression is limited to immune cells and endothelial cells. First, the effect of siRNA knockdown of TNFR1 in lung cancer cell lines was examined. siRNA knockdown of TNFR1 leads to inhibition of erlotinib induced NF-κB activation in both EGFR mutant and EGFRwt cells. Etanercept (Enbrel) is a fusion protein of TNFR and IgG1 and is in clinical use as a stable and effective TNF blocking agent for autoimmune diseases. Enbrel also blocks erlotinib induced NF-κB activation in multiple cell lines. Thalidomide, a drug that is known to reduce TNF levels, was also used. Thalidomide also inhibited erlotinib-induced NF-κB activation in both EGFRwt and EGFR mutant cell lines. It was confirmed that thalidomide inhibits erlotinib induced TNF increase in lung cancer cells. It should be noted that thalidomide is also reported to inhibit NF-κB activation independent of its effect on TNF. Consistent with this effect, it was found that thalidomide can block NF-κB activation induced by exogenous TNF. Thus, these studies indicate that erlotinib induces activation of NF-κB via increased TNF signaling.

It was recently found that EGFR inhibition results in activation of other signals such as JNK and ERK activation in glioma cells. However, in lung cancer cells, and consistent with what has been reported previously, although these signals are attenuated following EGFR inhibition, neither ERK nor JNK re-activation is detected.

### e. Erlotinib Induced TNF Expression is Regulated by NF-κB in a Feedforward Loop

TNF is an inducible cytokine and is regulated at multiple levels including transcription. NF-κB is a key transcription factor involved in TNF transcription. The possibility that erlotinib-induced increase in TNF expression may also be mediated by NF-κB in a feedforward loop was considered. Whether inhibition of NF-κB using a chemical inhibitor, or a dominant negative IkBα (super repressor) mutant would block the increase in TNF following exposure of cells to erlotinib was examined. Indeed, it was found that inhibition of NF-κB blocks the erlotinib-induced increase in TNF mRNA as detected by quantitative real time PCR. NF-κB activity is essential for TNF upregulation in both EGFRwt as well as EGFR mutant cell lines. As an additional negative control, Mithramycin was used as an inhibitor of Sp1. Although Sp1 binding sites are present in the TNF promoter, there is no effect of Sp1 inhibition on erlotinib-induced TNF upregulation.

Next, whether NF-κB and TNF induce each other in a feedforward loop was examined. If this is the case, then it should be possible to inhibit erlotinib-induced TNF upregulation by inhibition of the TNFR. Indeed, it was found that blocking the TNFR1 using siRNA or Etanercept results in inhibition of erlotinib-induced TNF upregulation. Without wishing to be bound by theory, these data indicate that TNF is upregulated via a feedforward loop that includes activity of NF-κB and TNFR1 signaling.

Finally, it was found that NF-κB can bind to two putative sites on the TNF promotor by ChIP-qPCR assay. It was shown that NF-κB can be detected on the TNF promotor by ChIP in cells. While there is some binding of NF-κB to the TNF promoter even under basal conditions, when EGFR is inhibited there is increased presence of NF-κB on the TNF promoter in both EGFRwt and EGFR mutant cells.

### f. TNF Protects Lung Cancer Cells from EGFR Inhibition

The TNF level is unregulated by EGFR inhibition using tyrosine kinase inhibitors in all 18 lung cancer cell lines and in the animal models that were tested. This led to an investigation of whether the TNF upregulation has biological significance. In particular, it was hypothesized that increased TNF secretion protects EGFR expressing lung cancer cells from cell death following the loss of EGFR signaling. A549 and H441 cell lines were used, which express EGFRwt and are known to be resistant to EGFR TKIs. First, siRNA knockdown of TNFR1 was done, and it was found that this confers sensitivity to erlotinib in cell survival assays. Erlotinib alone or TNFR1 silencing alone has no effect on the viability of these cells. Next, the effect of thalidomide, an inhibitor of TNF and of NF-κB activation, was examined. Thalidomide alone had no effect, but it rendered A549 and H441 cells sensitive to the effects of erlotinib. Thus, EGFR inhibition combined with either biological or chemical inhibition of TNF signaling renders EGFRwt expressing resistant cells sensitive to EGFR inhibition. Etanercept (Enbrel) also rendered both A549 and H441 cells sensitive to the effect of erlotinib, whereas Etanercept alone had no effect. The ability of the combination of Etanercept or thalidomide with erlotinib or afatinib (1 μM each) to impact cell viability was also examined. In fact, a statistically significant Enbrel or thalidomide sensitizing effect was observed if the EGFR inhibitor concentration was decreased to 100 nM.

Next, the effect of combining TNF and EGFR inhibition in lung cancer cells (HCC827, EGFR exon 19 deletion, or H3255, EGFR L858R mutation) that are oncogene addicted and sensitive to EGFR inhibition was investigated. Experiments with low concentrations of erlotinib revealed a sensitizing effect of TNF inhibition obtained by TNFR1 gene silencing. A combination of erlotinib and thalidomide also enhanced the sensitivity of HCC827 and H3255 cells to EGFR inhibition. Similarly, a combination of erlotinib and Enbrel results in greater sensitivity to EGFR inhibition in HCC827 and H3255 cells. TNF inhibition alone had no effect on the viability of oncogene-addicted cells. A combination of afatinib and thalidomide or Enbrel was also examined, and a greater sensitivity to EGFR inhibition was found.

Additional NSCLC lines with EGFRwt (Calu-3 and H1373) exhibited similar results with combined inhibition. In addition, H1975 cells (with a T790M mutation) were tested using afatinib, and it was found that these cells also can be rendered sensitive to EGFR inhibition if TNFR is inhibited.

Since it was hypothesized that erlotinib-induced TNF expression mediates resistance to EGFR inhibition, whether exogenous TNF would protect cells from erlotinib-induced cell death was examined. This experiment was conducted in EGFR oncogene addicted mutant cell lines, since EGFRwt cell lines are resistant to erlotinib alone. Indeed, it was found that exogenous TNF protects HCC827 and H3255 cells from erlotinib-induced cell death.

### g. Inhibition of NF-κB Results Enhances Sensitivity to EGFR Inhibition

NF-κB is a key component of inflammation-induced cancer. Previous studies have shown that NF-κB plays a role in resistance to EGFR inhibition in EGFR mutant cells. Here, the data indicate that the activation of NF-κB by EGFR inhibition is not limited to cells with EGFR activating mutations and is also detected in NSCLC cells with EGFRwt. Whether inhibition of NF-κB would sensitize lung cancer cells with EGFRwt to the effects of EGFR inhibition was examined. Indeed, it was found that inhibition of NF-κB using either two different inhibitors rendered two EGFRwt expressing cell lines sensitive to EGFR inhibition. It was also confirmed that inhibition of NF-κB enhanced sensitivity of oncogene addicted cells to EGFR inhibition, consistent with previous reports. Finally, it was found that overexpressing the p65 subunit of NF-κB results in a resistance to combined exposure of lung cancer cells to EGFR and TNF inhibition, suggesting that TNF-induced sensitization to EGFR inhibition is mediated, at least in part, via NF-κB activation.

### h. A Combined Inhibition of TNF and EGFR in an Animal Model of Lung Cancer

Next, it was examined whether a combined inhibition of TNF and EGFR would influence sensitivity to erlotinib in a mouse xenograft model. The experiments were started with the A549 cell line that expresses EGFRwt and is resistant to EGFR inhibition. Since studies indicate that a TNF-NF-κB loop is a key mediator of resistance to EGFR inhibition, thalidomide was chosen for the initial studies. A number of studies have demonstrated that thalidomide downregulates TNF levels and also inhibits NF-κB activation directly. A549 cells were injected into the flanks of mice to form subcutaneous tumors. Once tumors became visible, treatment was started with control vehicle, erlotinib, thalidomide, or erlotinib plus thalidomide. As expected, robust tumor growth was found in controls. The Erlotinib and thalidomide alone treated groups had a minor decrease in tumor growth that was not statistically significant. However, a combined inhibition of erlotinib and thalidomide resulted in a highly effective suppression of tumor growth. Next, the effect of EGFR+TNF inhibition was examined using thalidomide in an EGFRwt NSCLC patient derived xenograft tumor. The combination of erotinib+thalidomide was highly effective in inhibiting the growth of this PDX tumor. Additionally, the effect of a combined TNF and EGFR inhibition was examined in a mouse subcutaneous model using EGFR mutant erlotinib sensitive HCC827 cells, and it was found that the combination of EGFR inhibition plus thalidomide results in a more effective inhibition of tumor growth than EGFR inhibition alone while thalidomide alone had no significant effect. Next, to definitively determine the role of TNF, the effect of stably silencing TNF was examined using shRNA. Effective silencing of TNF was determined by decreased basal level and a lack of TNF upregulation in response to LPS by qPCR and ELISA. It was also confirmed that TNF silenced clones were more sensitive to EGFR inhibition in cell viability assays. Next, the effect of EGFR inhibition was determined in A549 cells with stably silenced TNF in a mouse subcutaneous model. Stable silencing of TNF results in enhanced sensitivity of xenografted tumors to erlotinib. Next, the effect of a specific TNF blocker, Etanercept, which is in clinical use, was examined. Again, it was found that Etanercept rendered A549 cells sensitive to the effect of EGFR inhibition.

Erlotinib in combination with either thalidomide or prednisone was effective to reduce tumor volume in an A549 EGRF wild type (EGFRwt) xenograft model relative to the use of these agents alone. Prednisone was shown to be more effective than thalidomide in combination with erlotinib for reducing tumor volume. The pharmaceutical composition combination of erlotinib and prednisone was more effective at reducing tumor volume than either of these agents used alone.

The pharmaceutical composition combination of erlotinib and prednisone is effective to shrink tumor volume beginning at day 32 in the A549 xenograft model.

The effect of withdrawing treatment of the A549 xenograft model with the combination of erlotinib and prednisone at day 32 versus maintaining treatment with this combination was evaluated. It is evident that tumor volume increases comparable to control with the combination therapy is withdrawn, whereas tumor volume shrinks if the combination therapy is continuously maintained.

Afatinib in combination with either thalidomide or prednisone was effective to reduce tumor volume in an H441 EGRF wild type (EGFRwt) xenograft model relative to the use of these agents alone. Prednisone is shown to be more effective than thalidomide in combination with erlotinib for reducing tumor volume. The pharmaceutical composition combination of afatinib and prednisone was more effective at reducing tumor volume than either of these agents used alone.

Afatinib in combination with either thalidomide or prednisone was effective to reduce tumor volume in an H1975 EGRF L858R/T790M xenograft model relative to the use of these agents alone. Both prednisone and thalidomide were found to be relatively equally effective in combination with erlotinib for reducing tumor volume. The pharmaceutical composition combination of afatinib and prednisone was more effective at reducing tumor volume than either of these agents used alone.

Prednisone is able to block the TNF upregulation that is induced by EGFR inhibition in both A549 and H441 cells.

### i. Isoniazid (INH) in Combination with EGFR Inhibition can Block the Growth of Tumors

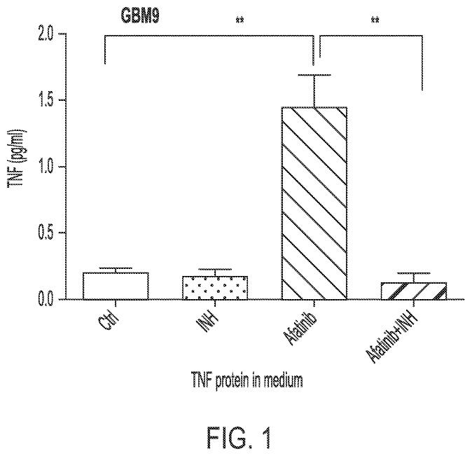

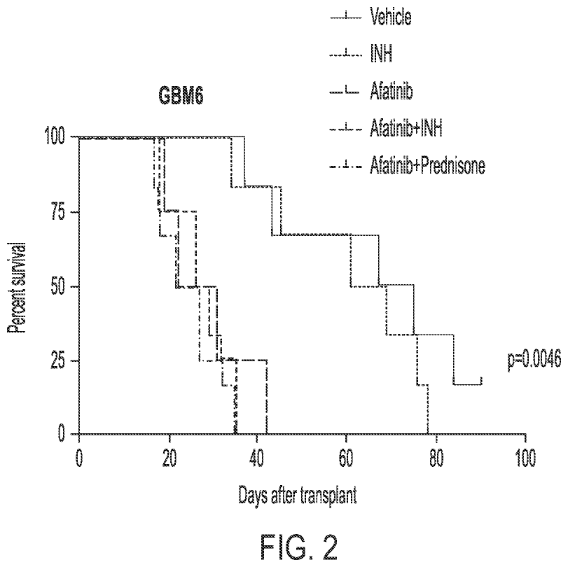

It was found that the drug Isoniazid (INH) can block TNF release (FIG. 1) and also found that EGFR inhibitors such as erlotinib or afatinib in combination with INH can block the growth of animal tumors as shown in FIG. 2. Thus, a combination of EGFR inhibitor plus INH is provided herein as useful in treatment of cancer.

As set forth in FIG. 1, brain cancer cells (GBM9) were treated with EGFR inhibitor (afatinib). Afatinib treatment leads to increased TNF secretion. The increased TNF secretion in response to afatinib is blocked (or inhibited or suppressed) by INH.

In another experiment, mice were injected with GBM6 brain tumor cells intracranially. After 10 days, tumors have formed and mice were divided into five groups followed by treatment as indicated. FIG. 2 shows a Kaplan-Meier survival curve. As set forth in FIG. 2, mice treated with a combination of afatinib+INH or afatinib+prednisone survive longer than other groups of mice.

### F. Equivalents

Those skilled in the art will recognize, or be able to ascertain using no more than routine experimentation, numerous equivalents to the specific procedures described herein. Such equivalents are considered to be within the scope of the present invention and are covered by the following claims. The contents of all references, patents, and patent applications cited throughout this application are hereby incorporated by reference. The appropriate components, processes, and methods of those patents, applications and other documents may be selected for the present invention and embodiments thereof.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for treating cancer in a patient in need thereof, the method comprising administering to the patient an effective amount of an agent that inhibits EGFR signaling, or a pharmaceutically acceptable salt thereof, and isoniazid (INH), or a pharmaceutically acceptable salt thereof.

2. The method of claim 1, wherein the agent that inhibits EGFR signaling is selected from the group consisting of erlotinib, afatinib, Cetuximab, panitumumab, and Gefitinib.

3. The method of claim 1, wherein the agent that inhibits EGFR signaling and isoniazid are co-formulated.

4. The method of claim 1, wherein the agent that inhibits EGFR signaling and isoniazid are co-packaged.

5. The method of claim 1, wherein the agent that inhibits EGFR signaling and isoniazid are administered concurrently.

6. The method of claim 1, wherein the agent that inhibits EGFR signaling and isoniazid are not administered concurrently.

7-8. (canceled)

9. The method of claim 1, wherein the effective amount is an individually effective amount of the agent that inhibits EGFR signaling or isoniazid.

10. The method of claim 1, wherein the effective amount is a combinatorically effective amount of the agent that inhibits EGFR signaling and isoniazid.

11-13. (canceled)

14. The method of claim 1, wherein the cancer is selected from the group consisting of brain cancer, lung cancer, cervical cancer, ovarian cancer, cancer of the central nervous system (CNS), skin cancer, prostate cancer, sarcoma, breast cancer, leukemia, colorectal cancer, colon cancer, head cancer, neck cancer, endometrial and kidney cancer.

15. The method of claim 1, wherein the cancer is lung cancer.

16. The method of claim 15, wherein the lung cancer is non-small cell lung cancer.

17. The method of claim 1, wherein the cancer is a human epithelial carcinoma.

18. The method of claim 17, wherein the human epithelial carcinoma is selected from the group consisting of basal cell carcinoma, squamous cell carcinoma, renal cell carcinoma (RCC), ductal carcinoma in situ (DCIS), and invasive ductal carcinoma.

19. The method of claim 1, wherein the cancer expresses EGFR wild type.

20. The method of claim 1, wherein the cancer expresses EGFR that contains at least one EGFR activating mutation.

21. The method of claim 1, wherein the cancer is resistant to EGFR inhibition.

22. A pharmaceutical composition comprising:
(a) an agent that inhibits EGFR signaling, or a pharmaceutically acceptable salt thereof;
(b) isoniazid, or a pharmaceutically acceptable salt thereof; and
(c) a pharmaceutically acceptable carrier,
wherein at least one of the agent that inhibits EGFR signaling and isoniazid is present in an effective amount.

23. The composition of claim 22, wherein the agent that inhibits EGFR signaling is selected from the group consisting of erlotinib, afatinib, Cetuximab, panitumumab, Erlotinib HCl, Gefitinib, Lapatinib, Neratinib, Lifirafenib, HER2-nhibitor-1, Nazartinib, Naquotinib, Canertinib, Lapatinib, AG-490, CP-724714, Dacomitinib, WZ4002, Sapitinib, CUDC-101, AG-1478, PD153035 HCL, pelitinib, AC480, AEE788, AP26113-analog, OSI-420, WZ3146, WZ8040, AST-1306, Rociletinib, Genisten, Varlitinib, Icotinib, TAK-285, WHI-P154, Daphnetin, PD168393, Tyrphostin9, CNX-2006, AG-18, AZ5104, Osimertinib, CL-387785, Olmutinib, AZD3759, Poziotinib, vandetanib, and necitumumab.

24. (canceled)

25. The composition of claim 22, wherein the agent that inhibits EGFR signaling is erlotinib.

26. A method for making a pharmaceutical composition, the method comprising combining:
(d) an agent that inhibits EGFR signaling, or a pharmaceutically acceptable salt thereof;
(e) isoniazid, or a pharmaceutically acceptable salt thereof; and
(f) a pharmaceutically acceptable carrier,
wherein at least one of the agent that inhibits EGFR signaling and isoniazid is present in an effective amount.

27-44. (canceled)
